(19) United States
(12) Patent Application Publication
(10) Pub. No.: US 2012/0129895 A1
(43) Pub. Date: May 24, 2012

(54) METHODS OF TREATMENT

(76) Inventors: Colleen Conway, Arlington, MA (US); Brant Geoffrey Firestone, Hyde Park, MA (US); Leigh Zawel, Hingham, MA (US)

(21) Appl. No.: 13/388,135

(22) PCT Filed: Aug. 11, 2010
(86) PCT No.: PCT/US2010/045111

§ 371 Date: Jan. 31, 2012

Related U.S. Application Data
(60) Provisional application No. 61/232,894, filed on Aug. 11, 2009.

(51) Int. Cl. A61K 31/427; A61P 35/00; A61K 31/352

(52) U.S. Cl. 514/365; 514/455

(57) ABSTRACT

The invention relates to combinations comprising a vascular disrupting agent (VDA) with IAP antagonists, for simultaneous, concurrent, separate or sequential use, especially for use in the treatment of proliferative diseases.

### FIELD OF INVENTION

The invention relates to combinations comprising a vascular disrupting agent (VDA) with Inhibitor of Apoptosis Proteins (IAP) antagonists, for simultaneous, separate or sequential use, especially for use in the treatment of proliferative diseases, more specifically cancer. The invention also relates to pharmaceutical compositions and commercial packages comprising such combinations and to methods for treating cancer, in a mammal, particularly a human, with such combinations.

### BACKGROUND OF INVENTION

(S)—N—((S)-1-cyclohexyl-2-{(S)-2-[4-(4-fluoro-benzoyl)-thiazol-2-yl]-pyrrolidin-1-yl}-2-oxo-ethyl)-2-methylamino-propionamide is a pan-IAP inhibitor developed as an anti-cancer agent capable of inducing apoptosis in tumor cells. Recently, in vitro studies have demonstrated that TNF, produced in an autocrine loop or provided exogenously, significantly enhances (S)—N—((S)-1-cyclohexyl-2-{(S)-2-[4-(4-fluoro-benzoyl)-thiazol-2-yl]-pyrrolidin-1-yl}-2-oxo-ethyl)-2-methylamino-propionamide-potentiated apoptosis i(Gaither A, 2007). 5,6-dimethylxanthenone-4-acetic acid is a vascular disrupting agent that has been reported to increase TNF mRNA in tumor, spleen, and liver of Colon 38 tumor bearing mice (Joseph W R, 1999).

### SUMMARY OF INVENTION

To leverage the TNF-inducing properties of 5,6-dimethylxanthenone-4-acetic acid, the combination of (S)—N—((S)-1-cyclohexyl-2-{(S)-2-[4-(4-fluoro-benzoyl)-thiazol-2-yl]-pyrrolidin-1-yl}-2-oxo-ethyl)-2-methylamino-propionamide and 5,6-dimethylxanthenone-4-acetic acid were investigated. Degradation of CIAP1 by IAP antagonists, including (S)—N—((S)-1-cyclohexyl-2-{(S)-2-[4-(4-fluoro-benzoyl)-thiazol-2-yl]-pyrrolidin-1-yl}-2-oxo-ethyl)-2-methylamino-propionamide, has sensitized many tumor cell lines to killing with TNF. The observation that 5,6-dimethylxanthenone-4-acetic acid induces the expression of TNF in host tumors and tissue provides a rationale for evaluating the combination of these two agents in the clinic. This property may lead to an increased population of cancer patients benefiting from 5,6-dimethylxanthenone-4-acetic acid and (S)—N—((S)-1-cyclohexyl-2-{(S)-2-[4-(4-fluoro-benzoyl)-thiazol-2-yl]-pyrrolidin-1-yl}-2-oxo-ethyl)-2-methylamino-propionamide therapy.

This invention relates to the combination of certain VDAs with IAP antagonists for the delay of progression or treatment of a proliferative disease, especially breast cancer, lung cancer or melanoma.

In a further aspect the invention provides use of a VDA in combination with an IAP antagonist for the treatment of a proliferative disease, especially breast cancer, lung cancer or melanoma.

In yet a further aspect the invention provides a VDA as active ingredient for use in combination with an IAP antagonist for the treatment of a proliferative disease, especially breast cancer or melanoma.

In still yet a further aspect, the invention provides a package comprising a VDA together with instructions for the use in combination with an IAP antagonist for the treatment of a proliferative disease, especially breast cancer, lung cancer or melanoma.

In still yet further aspect the invention provides single agent therapy of a vascular disrupting agent (VDA), especially 5,6-dimethylxanthenone-4-acetic acid, for the treatment of breast cancer.

In still yet a further aspect, the invention provides single agent therapy of IAP antagonists, especially (S)—N—((S)-1-cyclohexyl-2-{(S)-2-[4-(4-fluoro-benzoyl)-thiazol-2-yl]-pyrrolidin-1-yl}-2-oxo-ethyl)-2-methylamino-propionamide, for the treatment of breast cancer.

### DETAILED DESCRIPTION OF THE DRAWINGS

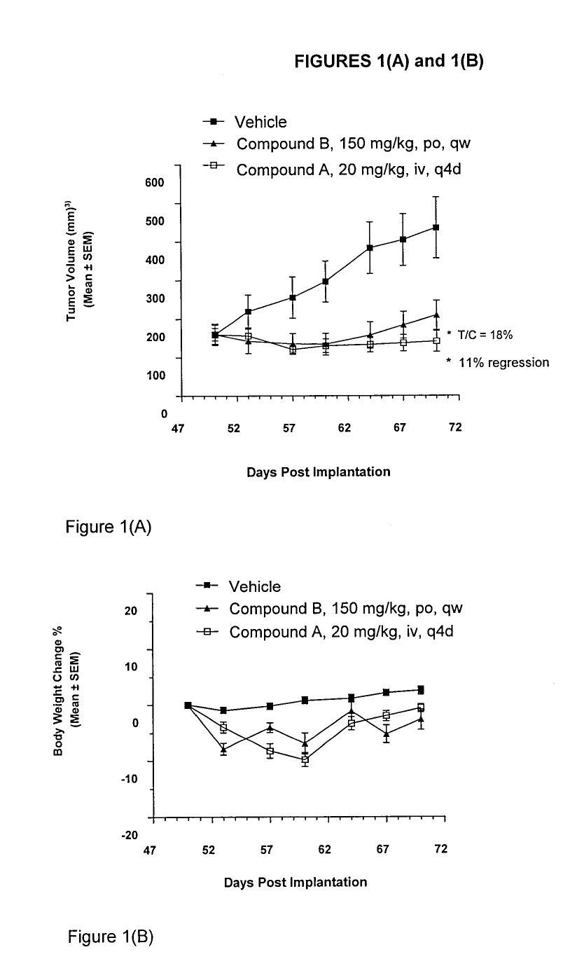

FIGS. 1(A) and 1(B) illustrate single agent anti-tumor activity of 5,6-dimethylxanthenone-4-acetic acid (Compound A) and (S)—N—((S)-1-cyclohexyl-2-{(S)-2-[4-(4-fluoro-benzoyl)-thiazol-2-yl]-pyrrolidin-1-yl}-2-oxo-ethyl)-2-methylamino-propionamide (Compound B) using a MDA-MB-231 human breast xenograft.

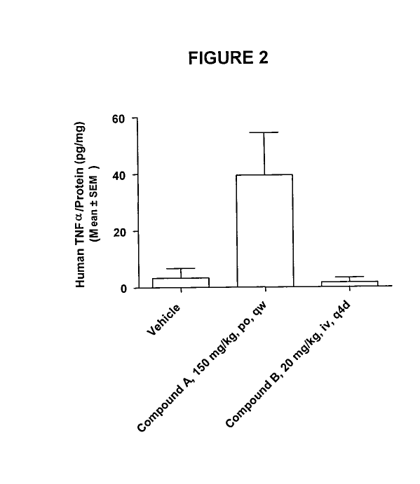

FIGS. 2(A) and 2(B) illustrate an increase in human tumor necrosis factor (TNF) production after treatment with (S)—N—((S)-1-cyclohexyl-2-{(S)-2-[4-(4-fluoro-benzoyl)-thiazol-2-yl]-pyrrolidin-1-yl}-2-oxo-ethyl)-2-methylamino-propionamide in breast xenograft tissue.

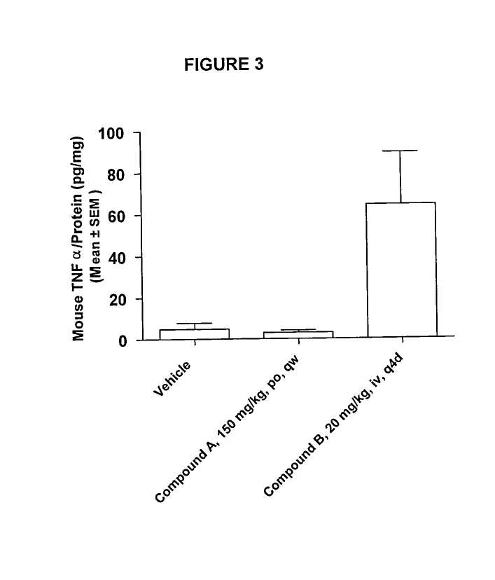

FIG. 3 illustrates an increase in mouse TNF production after treatment with 5,6-dimethylxanthenone-4-acetic acid in breast xenograft tissue.

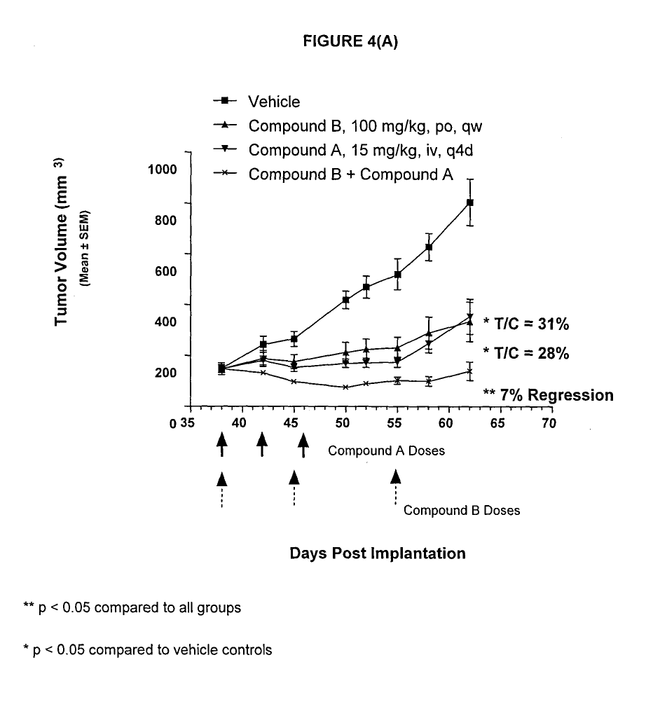

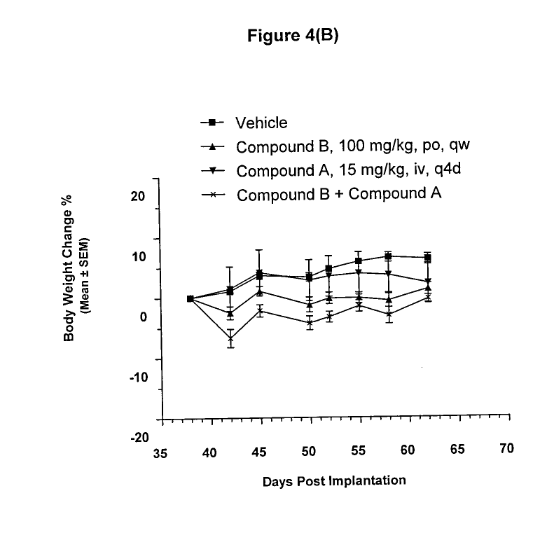

FIGS. 4(A) and 4(B) illustrate combination activity of (S)—N—((S)-1-cyclohexyl-2-{(S)-2-[4-(4-fluoro-benzoyl)-thiazol-2-yl]-pyrrolidin-1-yl}-2-oxo-ethyl)-2-methylamino-propionamide and 5,6-dimethylxanthenone-4-acetic acid in MDA-MB-231 xenograft.

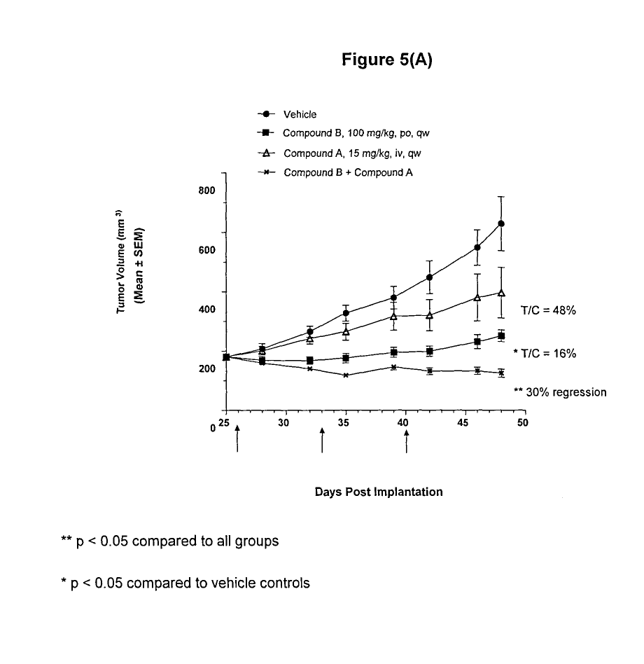

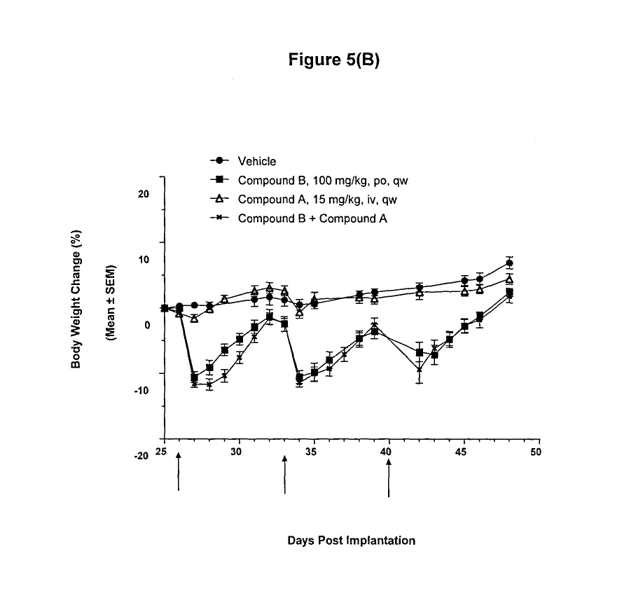

FIGS. 5(A) and 5(B) illustrate the combination activity of concurrent dosing of (S)—N—((S)-1-cyclohexyl-2-{(S)-2-[4-(4-fluoro-benzoyl)-thiazol-2-yl]-pyrrolidin-1-yl}-2-oxo-ethyl)-2-methylamino-propionamide and 5,6-dimethylxanthenone-4-acetic acid in MDA-MB-231 breast xenograft.

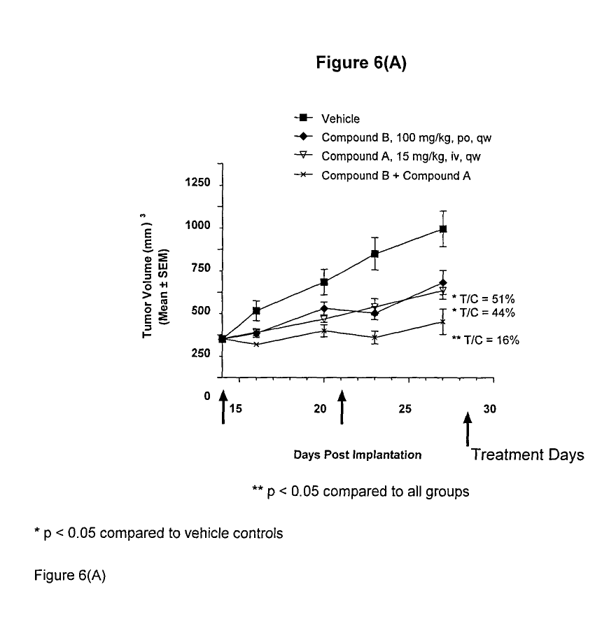

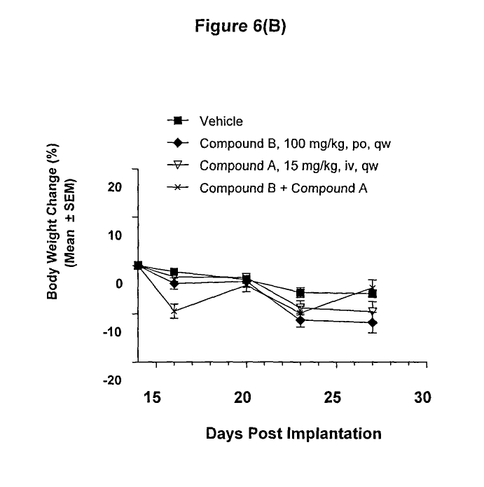

FIGS. 6(A) and 6(B) illustrate combination activity following treatment with (S)—N—((S)-1-cyclohexyl-2-{(S)-2-[4-(4-fluoro-benzoyl)-thiazol-2-yl]-pyrrolidin-1-yl}-2-oxo-ethyl)-2-methylamino-propionamide and 5,6-dimethylxanthenone-4-acetic acid in HS944.t human melanoma xenograft in nude mice.

### DETAILED DESCRIPTION OF INVENTION

Accordingly the invention provides a method for the delay of progression or treatment of breast cancer in a subject in need of such treatment which comprises administering to the subject an effective amount of a vascular disrupting agent

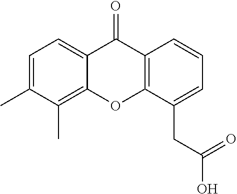

having the following formula (A):
or a pharmaceutically acceptable salt thereof. The compound of formula (A) is also known as 5,6-dimethylxanthenone-4-acetic acid.

IAP antagonists of the present invention have the following formula I:

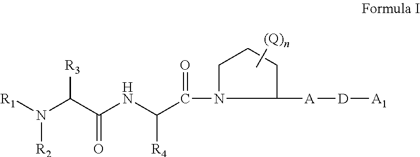

or pharmaceutically acceptable salts thereof, wherein

R₁ is H, C₁-C₄ alkyl, C₂-C₄ alkenyl, C₂-C₄ alkynyl or C₃-C₁₀ cycloalkyl, which R₁ may be unsubstituted or substituted;

R₂ is H, C₁-C₄ alkyl, C₂-C₄ alkenyl, C₂-C₄ alkynyl, C₃-C₁₀ cycloalkyl which R₂ may be unsubstituted or substituted;

R₃ is H, CF₃, C₂F₅, C₁-C₄ alkyl, C₂-C₄ alkenyl, C₂-C₄ alkynyl, CH₂—Z or R₂ and R₃ taken together with the nitrogen atom to which they are attached form a heterocyclic ring, which alkyl, alkenyl, alkynyl or het ring may be unsubstituted or substituted;

Z is H, OH, F, Cl, CH₃, CH₂Cl, CH₂F or CH₂OH;

R₄ is C₀₋₁₀ alkyl, C₃-C₁₀ cycloalkyl, wherein the C₀₋₁₀ alkyl, or cycloalkyl group is unsubstituted or substituted;

A is het, which may be substituted or unsubstituted;

D is C₁-C₇ alkylene or C₂-C₉ alkenylene, C(O), O, NR₇, S(O)r, C(O)—C₁-C₁₀ alkyl, O—C₁-C₁₀ alkyl, S(O)r-C₁-C₁₀ alkyl, C(O)C₀-C₁₀ arylalkyl OC₀-C₁₀ arylalkyl, or S(O)ᵣ C₀-C₁₀ arylalkyl, which alkyl and aryl groups may be unsubstituted or substituted;

r is 0, 1, or 2;

A₁ is a substituted aryl or unsubstituted or substituted het which substituents on aryl and het are halo, lower alkoxy, NR₅R₆, CN, NO₂ or SR₅;

Each Q is independently H, C₁-C₁₀ alkyl, C₁-C₁₀ alkoxy, aryl C₁-C₁₀ alkoxy, OH, O—C₁-C₁₀-alkyl, (CH₂)₀₋₆—C₃-C₇ cycloalkyl, aryl, aryl C₁-C₁₀ alkyl, O—(CH₂)₀₋₆ aryl, (CH₂)₁₋₆het, het, O—(CH₂)₁₋₆het, —OR₁₁, C(O)R₁₁, —C(O)N(R₁₁)(R₁₂), N(R₁₁)(R₁₂), SR₁₁, S(O)R₁₁,S(O)₂R₁₁, S(O)₂—N(R₁₁)(R₁₂), or NR₁₁—S(O)₂—(R₁₂), wherein alkyl, cycloalkyl and aryl are unsubstituted or substituted;

n is 0, 1, 2 or 3, 4, 5, 6 or 7;

“Het” is a 5-7 membered monocyclic heterocyclic ring containing 1-4 heteroring atoms selected from N,O and S or an 8-12 membered fused ring system that includes one 5-7 membered monocyclic heterocyclic ring containing 1, 2, or 3 heteroring atoms selected from N, O and S, which het is unsubstituted or substituted;

R₁₁ and R₁₂ are independently H, C₁-C₁₀ alkyl, (CH₂)₀₋₆—C₃-C₇cycloalkyl, (CH₂)₀₋₆—(CH)₀₋₁(aryl)₁₋₂,C(O)—C₁-C₁₀alkyl, —C(O)—(CH₂)₁₋₆—C₃-C₇cycloalkyl, —C(O)—O—(CH₂)₀₋₆-aryl, —C(O)—(CH₂)₀₋₆—O-fluorenyl, C(O)—NH—(CH₂)₀₋₆-aryl, C(O)—(CH₂)₀₋₆-aryl, C(O)—(CH₂)₁₋₆-het, —C(S)—C₁-C₁₀alkyl, —C(S)—(CH₂)₁₋₆—C₃-C₇cycloalkyl, —C(S)—O—(CH₂)₀₋₆-aryl, —C(S)—(CH₂)₀₋₆—O-fluorenyl, C(S)—NH—(CH₂)₀₋₆-aryl, —C(S)—(CH₂)₀₋₆-aryl or C(S)—(CH₂)₁₋₆-het, C(O)R₁₁, C(O)NR₁₁R₁₂, C(O)OR₁₁, S(O)nR₁₁, S(O)ₘNR₁₁R₁₂, m=1 or 2, C(S)R₁₁, C(S)NR₁₁R₁₂, C(S)OR₁₁, wherein alkyl, cycloalkyl and aryl are unsubstituted or substituted; or R₁₁ and R₁₂ are a substituent that facilitates transport of the molecule across a cell membrane; or R₁₁ and R₁₂ together with the nitrogen atom form het;

wherein the alkyl substituents of R₁₁ and R₁₂ may be unsubstituted or substituted by one or more substituents selected from C₁-C₁₀alkyl, halogen, OH, O—C₁-C₆alkyl, —S—C₁-C₆alkyl, CF₃ or NR₁₁R₁₂;

Substituted cycloalkyl substituents of R₁₁ and R₁₂ are substituted by one or more substituents selected from a C₂-C₁₀ alkene; C₁-C₆alkyl; halogen; OH; O—C₁-C₆alkyl; S—C₁-C₆alkyl, CF₃; or NR₁₁R₁₂ and

Substituted het or substituted aryl of R₁₁ and R₁₂ are substituted by one or more substituents selected from halogen, hydroxy, C₁-C₄ alkyl, C₁-C₄ alkoxy, nitro, CN O—C(O)—C₁-C₄alkyl and C(O)—O—C₁-C₄-alkyl;

R₅, R₆ and R₇ are independently hydrogen, lower alkyl, aryl, aryl lower alkyl, cycloalkyl, or cycloalkyl lower alkyl, and

wherein the substituents on R₁, R₂, R₃, R₄, Q, and A and A₁ groups are independently halo, hydroxy, lower alkyl, lower alkenyl, lower alkynyl, lower alkanoyl, lower alkoxy, aryl, aryl lower alkyl, amino, amino lower alkyl, diloweralkylamino, lower alkanoyl, amino lower alkoxy, nitro, cyano, cyano lower alkyl, carboxy, lower carbalkoxy, lower alkanoyl, aryloyl, lower arylalkanoyl, carbamoyl, N-mono- or N,N-dilower alkyl carbamoyl, lower alkyl carbamic acid ester, amidino, guanidine, ureido, mercapto, sulfo, lower alkylthio, sulfoamino, sulfonamide, benzosulfonamide, sulfonate, sulfanyl lower alkyl, aryl sulfonamide, halogen substituted aryl sulfonate, lower alkylsulfinyl, arylsulfinyl; aryl-lower alkylsulfinyl, lower alkylarylsulfinyl, lower alkylsulfonyl, arylsulfonyl, aryl-lower alkylsulfonyl, lower aryl alkyl lower alkylarylsulfonyl, halogen-lower alkylmercapto, halogen-lower alkylsulfonyl, phos-phono (—P(═O)(OH)₂), hydroxy-lower alkoxy phosphoryl or di-lower alkoxyphosphoryl, (R₉)NC(O)—NR₁₀R₁₃, lower alkyl carbamic acid ester or carbamates or —NR₈R₁₄, wherein R₈ and R₁₄ can be the same or different and are independently H or lower alkyl, or R₈ and R₁₄ together with the N atom form a 3- to 8-membered heterocyclic ring containing a nitrogen heteroring atoms and may optionally contain one or two additional heteroring atoms selected from nitrogen, oxygen and sulfur, which heterocyclic ring may be unsubstituted or substituted with lower alkyl, halo, lower alkenyl, lower alkynyl, hydroxy, lower alkoxy, nitro, amino, lower alkyl, amino, diloweralkyl amino, cyano, carboxy, lower carbalkoxy, formyl, lower alkanoyl, oxo, carbarmoyl, N-lower or N,N-dilower alkyl carbamoyl, mercapto, or lower alkylthio, and

R₉, R₁₀, and R₁₃ are independently hydrogen, lower alkyl, halogen substituted lower alkyl, aryl, aryl lower alkyl, halogen substituted aryl, halogen substituted aryl lower alkyl.

Further the invention provides the use of a compound of formula (I), or pharmaceutically acceptable salt or prodrug ester thereof, for the preparation of a medicament for use in combination with IAP antagonists in the treatment of a proliferative disease.

As used herein, the term “Aryl” is defined as an aromatic radical having 6 to 14 ring carbon atoms, and no ring heteroatoms The aryl group may be monocyclic or fused bicyclic or tricyclic. It may be unsubstituted or substituted by one or more, preferably one or two, substituents, wherein the substituents are as described herein. As defined herein, the aryl moiety may be completely aromatic regardless of whether it is monocyclic or bicyclic. However, if it contains more than one ring, as defined herein, the term aryl includes moieties wherein at least one ring is completely aromatic while the other ring(s) may be partially unsaturated or saturated or completely aromatic. Preferred “aryl” is phenyl, naphthyl or indanyl. The most preferred aryl is phenyl.

“Het” as used herein, refers to heteroaryl and heterocyclic compounds containing at least one S, O or N ring heteroatom. More specifically, “Het” is a 5-7 membered heterocyclic ring containing 1-4 heteroatoms selected from N, O and S, or an 8-12 membered fused ring system including at least one 5-7 membered heterocyclic ring containing 1, 2 or 3 heteroatoms selected from N, O, and S. Examples of het, as used herein, include unsubstituted and substituted pyrrolidyl, tetrahydrofuryl, tetrahydrothiofuryl, piperidyl, piperazyl, purinyl, tetrahydropyranyl, morpholino, 1,3-diazapanyl, 1,4-diazapanyl, 1,4-oxazepanyl, 1,4-oxathiapanyl, furyl, thienyl, pyrryl, pyrrolyl, pyrazolyl, triazolyl, tetrazolyl, indazolyl, oxadiazolyl, imidazolyl, pyrrolidyl, pyrrolidinyl, thiazolyl, oxazolyl, pyridyl, pyrazolyl, pyrazinyl, pyrimidinyl, isoxazolyl, pyrazinyl, quinolyl, isoquinolyl, pyridopyrazinyl, pyrrolopyridyl, furopyridyl, indolyl, benzofuryl, benzothiofuryl, benzoindolyl, benzothienyl, pyrazolyl, piperidyl, piperazinyl, indolinyl, morpholinyl, benzoxazolyl, pyrroloquinolyl, and the like. Heteroaryls are within the scope of the definition of het. Examples of heteroaryls are pyridyl, pyrimidinyl, quinolyl, thiazolyl and benzothiazolyl. The most preferred het are pyridyl, pyrimidinyl and thiazolyl. The het may be unsubstituted or substituted as described herein. It is preferred that it is unsubstituted or if substituted it is substituted on a carbon atom by halogen, especially fluorine or chlorine, hydroxy, C₁-C₄ alkyl, such as methyl and ethyl, C₁-C₄ alkoxy, especially methoxy and ethoxy, nitro, —O—C(O)—C₁-C₄alkyl or —C(O)—O—C₁-C₄alkyl, SCN or nitro or on a nitrogen atom by C₁-C₄ alkyl, especially methyl or ethyl, —O—C(O)—C₁-C₄alkyl or —C(O)—O—C₁-C₄alkyl, such as carbomethoxy or carboethoxy.

When two substituents together with a commonly bound nitrogen are het, it is understood that the resulting heterocyclic ring is a nitrogen-containing ring, such as aziridine, azetidine, azole, piperidine, piperazine, morphiline, pyrrole, pyrazole, thiazole, oxazole, pyridine, pyrimidine, isoxazole, and the like, wherein such het may be unsubstituted or substituted as defined hereinabove.

Halogen is fluorine, chlorine, bromine or iodine, especially fluorine and chlorine.

Unless otherwise specified “alkyl”, either above or in combination, includes straight or branched chain alkyl, such as methyl, ethyl, n-propyl, isopropyl, n-butyl, sec-butyl, tert-butyl, n-pentyl and branched pentyl, n-hexyl and branched hexyl, and the like.

A “cycloalkyl” group means C₃ to C₁₀ cycloalkyl having 3 to 10 ring carbon atoms and may be, for example, cyclopropyl, cyclobutyl, cyclopentyl, cyclohexyl, cycloheptyl or cyclooctyl, cyclononyl and the like. The cycloalkyl group may be monocyclic or fused bicyclic. It is preferred that it is monocyclic. Moreover, the preferred cycloalkyl group is cyclopentyl or cyclohexyl. Most preferably, cycloalkyl is cyclohexyl. The cycloalkyl group may be fully saturated or partially unsaturated, although it is preferred that it is fully saturated. As defined herein, it excludes aryl groups. The cycloalkyl groups may be unsubstituted or substituted with any of the substituents defined below, preferably halo, hydroxy or C₁-C₆ alkyl such as methyl.

Substituents that facilitate transport of the molecule across a cell membrane are known to those of skill in the medicinal chemistry arts (see, for example, Gangewar S., Pauletti G. M., Wang B., Siahaan T. J., Stella V. J., Borchardt R. T., Drug Discovery Today, vol. 2. p 148-155 (1997) and Bundgaard H. and Moss J., Pharmaceutical Research, vol. 7, p 885 (1990)). Generally, such substituents are lipophilic substituents. Such lipophilic substituents include a C₆-C₃₀ alkyl which is saturated, monounsaturated, polyunsaturated, including methylene-interrupted polyene, phenyl, phenyl which is substituted by one or two C₁-C₈ alkyl groups, C₅-C₉ cycloalkyl, C₅-C₉ cycloalkyl which is substituted by one or two C₁-C₈ alkyl groups, —X₁-phenyl, —X₁-phenyl which is substituted in the phenyl ring by one or two C₁-C₈ alkyl groups, X₁—C₅-C₉ cycloalkyl or X₁—C₅-C₉ cycloalkyl which is substituted by one or two C₁-C₈ alkyl groups; where X₁ is C₁-C₂₄ alkyl which is saturated, monounsaturated or polyunsaturated and straight or branched chain.

Unsubstituted is intended to mean that hydrogen is the only substituent.

Except as described herein, any of the above defined aryl, het, alkyl, alkenyl, alkynyl, or cycloalkyl, may be unsubstituted or independently substituted by up to four, preferably one, two or three substituents, selected from the group consisting of: halo (such as Cl or Br); hydroxy; lower alkyl (such as C₁-C₃ alkyl); lower alkyl which may be substituted with any of the substituents defined herein; lower alkenyl; lower alkynyl; lower alkanoyl; lower alkoxy (such as methoxy); aryl (such as phenyl or naphthyl); substituted aryl (such as fluoro phenyl or methoxy phenyl); aryl lower alkyl such as benzyl, amino, mono or di-lower alkyl (such as dimethylamino); lower alkanoyl amino acetylamino; amino lower alkoxy (such as ethoxyamine); nitro; cyano; cyano lower alkyl; carboxy; lower carbalkoxy (such as methoxy carbonyl; n-propoxy carbonyl or iso-propoxy carbonyl), lower aryloyl, such as benzoyl; carbamoyl; N-mono- or N,N di-lower alkyl carbamoyl; lower alkyl carbamic acid ester; amidino; guanidine; ureido; mercapto; sulfo; lower alkylthio; sulfoamino; sulfonamide; benzosulfonamide; sulfonate; sulfanyl lower alkyl (such as methyl sulfanyl); sulfoamino; aryl sulfonamide; halogen substituted or unsubstituted aryl sulfonate (such as chloro-phenyl sulfonate); lower alkylsulfinyl; arylsulfinyl; aryl-lower alkylsulfinyl; lower alkylarylsulfinyl; lower alkanesulfonyl; arylsulfonyl; aryl-lower alkylsulfonyl; lower aryl alkyl; lower alkylarylsulfonyl; halogen-lower alkylmercapto; halogen-lower alkylsulfonyl; such as trifluoromethane sulfonyl; phosphono(—P(═O)(OH)₂); hydroxy-lower alkoxy phosphoryl or di-lower alkoxyphosphoryl; urea and substituted urea of the formula (R₉) N C(O)N(R₁₀), (R₁₃) wherein R₉, R₁₀ and R₁₃ are as defined herein (such as urea or 3-trifluoro-methyl-phenyl urea); alkyl carbamic acid ester or carbamates (such as ethyl-N-phenyl-carbamate) or —NR₈R₁₄, wherein R₈ and R₁₄ can be the same or different and are independently H; lower alkyl (e.g. methyl, ethyl or propyl); or R₈ and R₁₄ together with the N atom form a 3- to 8-membered heterocyclic ring containing a nitrogen heteroring atom and optionally one or two additional heteroring atoms selected from the group consisting of nitrogen, oxygen and sulfur (e.g. piperazinyl, pyrazinyl, lower alkyl-piperazinyl, pyridyl, indolyl, thiophenyl, thiazolyl, benzothiophenyl, pyrrolidinyl, piperidino or imidazolinyl) where the heterocyclic ring may be substituted with any of the substituents defined hereinabove.

Preferably the above mentioned alkyl, cycloalkyl, and aryl groups are independently unsubstituted or are substituted by lower alkyl, aryl, aryl lower alkyl, carboxy, lower carbalkoxy and especially halogen, —OH, —SH, —OCH₃, —SCH₃, —CN, —SCN or nitro.

As defined herein the term “lower alkyl”, when used alone or in combination refers to alkyl containing 1-6 carbon atoms. The alkyl group may be branched or straight-chained, and is as defined hereinabove.

The term “lower alkenyl” refers to an alkenyl group which contains 2-6 carbon atoms. An alkenyl group is a hydrocarbyl group containing at least one carbon-carbon double bond. As defined herein, it may be unsubstituted or substituted with the substituents described herein. The carbon-carbon double bonds may be between any two carbon atoms of the alkenyl group. It is preferred that it contains 1 or 2 carbon-carbon double bonds and more preferably one carbon-carbon double bond. The alkenyl group may be straight chained or branched. Examples include ethenyl, 1-propenyl, 2-propenyl, 1-butenyl, 2-butenyl, 2-methyl-1-propenyl, 1,3-butadienyl, and the like. The preferred alkenyl group is ethenyl.

The term “lower alkynyl”, as used herein, refers to an alkynyl group containing 2-6 carbon atoms. An alkynyl group is a hydrocarbyl group containing at least one carbon-carbon triple bond. The carbon-carbon triple bond may be between any two carbon atom of the alkynyl group. It is preferred that the alkynyl group contains 1 or 2 carbon-carbon triple bonds and more preferably one carbon-carbon triple bond. The alkynyl group may be straight chained or branched. Examples include ethynyl, 1-propynyl, 2-propynyl, 1-butynyl, 2-butynyl and the like. The preferred alkynyl group is ethynyl.

As used herein, the term “aryl alkyl” refers to a aryl group connected to the main chain by a bridging alkylene group. Examples include benzyl, phenethyl, naphthylmethyl, and the like. The preferred aryl alkyl is benzyl. Similarly, cyano alkyl group refers to a cyano group connected to the main chain by a bridging alkylene group.

The term “alkyl aryl” on the other hand, refers to an alkyl group bridged to the main chain through a phenylene group. Examples include methylphenyl, ethylphenyl, and the like.

As used herein, the term lower alkanoyl refers to a lower alkyl chain in which one of the carbon atoms is replaced by a C═O group. The C═O group may be present at one of the ends of the substituent or in the middle of the moiety. Examples include formyl, acetyl, 2-propanoyl, 1-propanoyl and the like.

The term “alkoxy” refers to an alkyl group as defined herein, connected to the main chain by an oxygen atom. Examples include methoxy, ethoxy, and the like.

The term “lower thioalkyl” refers to an alkyl group, as defined herein, connected to the main chain by a sulfur atom. Examples include thiomethyl (or mercapto methyl), thioethyl(mercapto ethyl) and the like.

The term “lower carbalkoxy” or synonym thereto refers to an alkoxycarbonyl group, where the attachment to the main chain is through the aryl group (C(O)). Examples include methoxy carbonyl, ethoxy carbonyl, and the like.

It is to be understood that the terminology C(O) refers to a —C═O group, whether it be ketone, aldehyde or acid or acid derivative. Similarly, S(O) refers to a —S═O group.

As used herein, the term S(O)r refers to the number of oxygen atoms bonded to the sulfur atom. When r=2, then S(O)ᵣ═SO₂; when r is 1, then S(O)r is SO; and when r=O, then S(O)r is S.

The term “Cₒ”, as used herein, as part of a definition of alkyl, as e.g., C₀₋₁₀, refers to zero carbon atoms. Thus, “C₀-C₁₀ aryl alkyl” means that the aryl group is bonded directly to the main chain (Cₒ) or that there is a C₁-C₁₀ alkylene group bridging the main chain to an aryl group.

The term “(CH₂)₀₋₆” as part of definition of a larger group, e.g., (CH₂)₀₋₆ C₃-C₇ cycloalkyl, refers to a group that is not present (CH₂)₀, or to a group that contains 1-6 carbon atoms (CH₂)₁₋₆.

The term (CH₂)₀₋₆—(CH)₀₋₁, (aryl)₁₋₂, in the definition of R₁₁ and R₁₂, is intended to mean one of the following (CH₂)₁₋₆-aryl, aryl, —CH(aryl)₂ or (CH₂)₁₋₆ (CH) (aryl)₂.

As used herein, the variable n refers to number of substituents on the pyrrolidinyl(tetrahydropyrrolyl) ring. The term “n” is defined as 0-7 and it determines the number of Q substituents on the pyrrolidinyl(tetrahydro-pyrrolyl) ring. Q can only be present at the 2, 3, 4, or 5 positions of the pyrrolidinyl ring, i.e., at the carbon atoms of the pyrrolidinyl ring. Except for carbon number 2 that can allow for one substitution, each of other carbon atoms are saturated and each of them may have two substituents thereon. When n is 7, then each of the carbon atoms are bonded with Q as defined herein. Each Q may be the same or different. However, when n is 6, then one of the seven possible substituents is H, and the other five are Q, which can be the same or different. Further, when n is 5, then two of the possible substituents are H, and the other five are independently Q, as defined herein. When n is 4, then three of the seven possible substituents are H, and the remainder are Q independently as defined herein. Where n is 3, then four of the seven possible substituents are H, and the other three are Q as defined herein. When n is 2, then two of the seven possible substituent are Q, and the remainder are H. When n is 1, then only one of the seven possible substituent is Q, and the remainder are H. Finally, when n is 0, all seven of the substituents are H.

It is to be understood that each of the Q substituents may be the same or they may be different.

Where the plural form is used for compounds, salts, pharmaceutical preparations, this is intended to mean also a single compound, single pharmaceutical preparation, salt, and the like.

It will be apparent to one of skill in the art that the compounds of the present invention can exist as a salt form, especially as an acid addition salt or a base addition salt. When a compound exists in a salt form, such salt forms are included within the scope of the invention. Although any salt form may be useful in chemical manipulations, such as purification procedures, only pharmaceutically acceptable salts are useful for the pharmaceutical products of the present invention.

Pharmaceutically acceptable salts include, when appropriate, pharmaceutically acceptable base addition salts and acid addition salts, for example, metal salts, such as alkali and alkaline earth metal salts, ammonium salts, organic amine addition salts, and amino acid addition salts, and sulfonate salts and the like. Acid addition salts include inorganic acid addition salts such as hydrochloride, sulfate and phosphate, and organic acid addition salts such as alkyl sulfonate, arylsulfonate, acetate, maleate, fumarate, tartrate, citrate and lactate and the like. Examples of metal salts are alkali metal salts, such as lithium salt, sodium salt and potassium salt, alkaline earth metal salts such as magnesium salt and calcium salt, aluminum salt, and zinc salt and the like. Examples of ammonium salts are ammonium salts and tetramethylammonium salts and the like. Examples of organic amine addition salts are salts with morpholine and piperidine and the like. Examples of amino acid addition salts are salts with glycine, phenylalanine, glutamic acid and lysine and the like. Sulfonate salts include mesylate, tosylate and benzene sulfonic acid salts and the like.

In view of the close relationship between the compounds in free form and those in the form of their salts, including those salts that can be used as intermediates, for example, in the purification or identification of the compounds, tautomers or tautomeric mixtures and their salts, any reference to the compounds hereinbefore and hereinafter especially the compounds of the Formula I-VII, is to be understood as referring also to the corresponding tautomers of these compounds, especially of compounds of the Formula I-VII, tautomeric mixtures of these compounds, especially of compounds of the Formula I-VII, or salts of any of these, as appropriate and expedient and if not mentioned otherwise.

Any asymmetric carbon atom may be present in the (R)-, (S)- or (R,S)-configuration, preferably in the (R)- or (S)-configuration. Substituents at a ring at atoms with saturated bonds or substituents on carbon-carbon double bonds may, if possible, be present in cis- (=Z-) or trans (=E-) form. The compounds may thus be present as mixtures of isomers or preferably as pure isomers, preferably as enantiomermally pure diastereomers or pure enantiomers.

The present invention includes within its scope, prodrugs of the compounds of the invention. In general, such prodrugs will be functional derivatives of a compound of the invention which are readily convertible in vivo into the compound from which it is notionally derived. Conventional procedures for the selection and preparation of suitable prodrugs are described, for example, in “Design of Prodrugs” ed. H. Bundgaard, Elsevier, 1985.

The preferred R₁ group is H and C₁-C₄ alkyl especially methyl. R₁ may be unsubstituted or substituted and is most preferably unsubstituted. The most preferred values of R₁ is H, methyl and ethyl, and especially methyl or ethyl and most especially methyl.

R₂ is preferably H or C₁-C₄ alkyl, especially methyl. R₂ may be unsubstituted or substituted. It is most preferably unsubstituted. It is preferred that R₂ is hydrogen.

R₃ is preferably H or C₁-C₄ alkyl especially hydrogen methyl, or ethyl and most especially methyl or ethyl, and most especially methyl, which may be unsubstituted or substituted. R₃ may be unsubstituted or substituted as defined herein. It is preferred that it is unsubstituted methyl.

R₄ is preferably C₅-C₇ cycloalkyl, and more preferably cyclopentyl or cyclohexyl, and most preferably is cyclohexyl. One may be substituted or unsubstituted. If substituted, it is preferably substituted with lower alkyl especially methyl. However, it is preferred that R₄ is unsubstituted.

The pyrrolidinyl ring can have up to six independent Q substituents thereon. It is preferred that n is 0-3 and even more preferably, n is 0, 1, or 2 and even more preferably, n is 0 or 1 and most preferably n is 0. If Q is present, it is preferred that Q is lower alkyl, alkoxyl, alylthio, amino, sulfonylamino, acylamino.

A is preferably a 5 or 6-membered het, and more preferably is 5 or 6 membered heteroaryl, especially a 5 or 6-member heteroaryl ring containing at least one ring hetero atom selected from the group consisting of nitrogen, oxygen and sulfur and containing 1-4 ring heteroatoms. Preferably, it contains 1 or 2 ring heteroatoms, and more preferably contains at least 1N ring heteroatom, and the other ring heteroatom is, if present, a nitrogen, oxygen or sulfur, and more preferably if present is nitrogen or sulfur, and most preferably, if present, is sulfur. The preferred value of A is pyridyl, pyrimidinyl, and thiazolyl. A may be unsubstituted or substituted. It is preferred that A is unsubstituted or substituted with alkyl, amino or halo.

D is preferably O or C(O)NR₇ or S(O)r and more preferably is O or C(O)HN or S and even more preferably is O or C(O) or HN and most preferably O or C(O).

A₁ is preferably a substituted aryl or an unsubstituted or substituted 5 or 6 membered het and more preferably is a substituted aryl or a 5 or 6 membered unsubstituted or substituted heteroaryl. Most preferably, A₁ is a substituted aryl. The most preferred value of A₁ is a substituted phenyl.

It is preferred that if A is aryl, it is monosubstituted, disubstituted or trisubstituted by one of the substituents enumerated hereinabove. In an embodiment, A₁ is substituted by halo, especially fluoro or chloro, and most preferably fluoro.

Another embodiment of the compound of Formula I has Formula II:

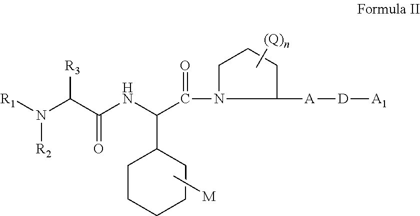

or pharmaceutically acceptable salts thereof, wherein

R₁, R₂, R₃, Q, n, A, D, and A₁ are as described hereinabove, and M is an H or a substituent on the cyclohexyl group, as defined above. It is preferred that M is H, halo; hydroxy; lower alkyl; lower alkenyl; lower alkynyl; formyl; lower alkanoyl; aryl, cycloalkyl, aryl lower alkyl, lower alkoxy; aryl lower alkyl, amino; amino, mono- or disubstituted lower alkylamino; amino lower alkyl; lower alkanoyl; amino lower alkoxy; nitro; cyano; cyano lower alkyl; carboxy; lower carbalkoxy; lower alkanoyl; arylyol; lower arylalkanoyl; carbamoyl; N-mono- or N,N-lower alkyl carbamoyl; lower alkyl carbamic acid ester; —NR₁₅R₁₆, wherein R₁₅ and R₁₆ can be the same or different and are independently H or lower alkyl; or R₁₅ and R₁₆ together with the N atom form a 3- to 8-membered heterocyclic ring containing 1-4 nitrogen, oxygen or sulfur ring atoms. The preferred value of M is hydrogen. The preferred values of R₁, R₂, R₃, n, Q, A, D, A₁, and Y described hereinabove are also applicable in this embodiment.

In an another embodiment, the compounds of Formula II have the Formula III

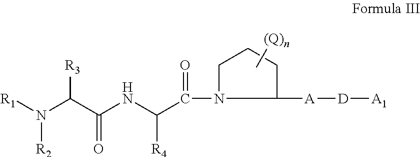

or pharmaceutically acceptable salts thereof wherein

R₁ is H or C₁-C₄ alkyl, which may be unsubstituted or substituted;

R₂ is H or C₁-C₄-alkyl, which may be unsubstituted or substituted;

R₃ is H or C₁-C₄ alkyl, which may be unsubstituted or substituted;

R₄ is cyclohexyl, which may be unsubstituted or substituted;

A is 5 or 6 membered nitrogen, oxygen or sulfur containing heteroaryl, which may be unsubstituted or substituted;

D is C(O), O, S(O)r or NR₇;

A₁ is aryl, which is substituted; and

n is as defined hereinabove.

The preferred definition of the various variables described hereinabove are also applicable in this embodiment. However, the preferred values of R₁ is H or methyl. In addition, the preferred value of R₂ is hydrogen or methyl. The preferred value of R₃ is hydrogen, ethyl or methyl.

It is preferred that n is 0, that is, the remaining substituents on the pyrrolidine ring are all hydrogens.

The preferred values of A is a 5 or 6 membered heteroaryl ring containing at least 1 nitrogen ring atom. Preferably, it contains 1 nitrogen ring atom and 4-5 carbon ring atoms or 2 ring heteroatoms and 3-4 ring carbon atoms, wherein 1 of the ring atoms is a nitrogen atom, and the other ring heteroatom is an oxygen, sulfur or nitrogen atom. Examples include pyridyl (e.g., 2-pyridyl, 3-pyridyl, or 4-pyridyl), pyrimidinyl, thiazolyl, oxazolyl and pyrrolyl (e.g., 2-pyrrolyl).

In an embodiment A₁ is substituted aryl, especially substituted phenyl, wherein the substituents are as defined hereinabove.

The preferred values of D is C(O), O, S, NR₁₁R¹² and most especially C(O) or O.

Another embodiment is a compound of Formula I has Formula IV:

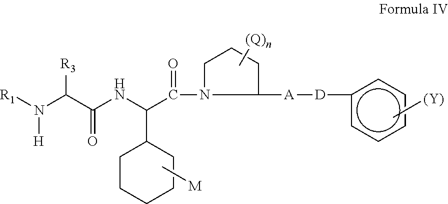

or pharmaceutically acceptable salts thereof,

wherein R₁, R₃, Q, n, A₁, D, M and are as defined hereinabove and “Y” is halo, lower alkoxy, NR₅R₆, CN, NO₂ or SR₅ wherein R₅ and R₆ are defined above. In an embodiment, Y is halo. It is preferred that Y is on the para position. The preferred value of R₁, R₃, Q, n, A₁ and D described hereinabove are also applicable in this embodiment. It is most preferred that Y is fluorine.

A further embodiment is directed to compounds of the formula:

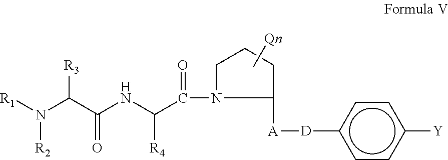

or pharmaceutically acceptable salts thereof
wherein R₂ is H, R₄ is unsubstituted cyclohexyl an R₁, R₃, A, D, and Y are as defined hereinabove. The various values of R₁, R₃, A, D, Qn and Y described hereinabove are also applicable. In the compound of Formula V, in an embodiment, as described hereinabove, n is 0.

In a preferred embodiment of the present invention, R₃ and R₄ have the stereochemistry indicated in Formula VI, with the definitions of the various substituents described hereinabove with respect to formulae I-V also applying to compounds of Formula VI. Thus, another embodiment of the present invention is directed to compounds of Formula VI

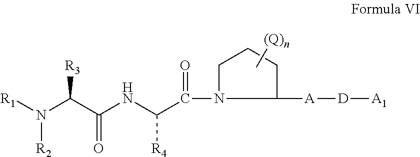

or pharmaceutically acceptable salts thereof
wherein R₁, R₂, R₃, R₄, Q, n, A, D and A₁, Y are as defined in any of the embodiments hereinabove.

In another embodiment of the present invention, the compound of Formula VI has the formula:

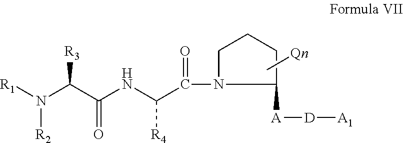

or pharmaceutically acceptable salts thereof wherein
R₁, R₂, R₃, R₄, A, D, Q and A₁, and n are as defined hereinabove.

It is to be understood that the preferred values of the variables described hereinabove with respect to compounds of Formulae I-V are also applicable to the compounds of Formula VI and VII.

The preferred compounds are selected from the group consisting of (S)—N—((S)-1-Cyclohexyl-2-{(S)-2-[4-(4-fluoro-benzoyl)-thiazol-2-yl]-pyrrolidin-1-yl}-2-oxo-ethyl)-2-methylamino-propionamide; (S)—N—[(S)-Cyclohexyl-(ethyl-{(S)-1-[5-(4-fluoro-benzoyl)-pyridin-3-yl]-propyl}carbamoyl)-methyl]-2-methylamino-propionamide; and (S)—N—((S)-1-Cyclohexyl-2-{(S)-2-[5-(4-fluoro-phenoxy)-pyridin-3-yl]-pyrrolidin-1-yl}-2-oxo-ethyl)-2-methylamino-propionamide; and pharmaceutically acceptable salts thereof.

(S)—N—((S)-1-Cyclohexyl-2-{(S)-2-[4-(4-fluoro-benzoyl)-thiazol-2-yl]-pyrrolidin-1-yl}-2-oxo-ethyl)-2-methylamino-propionamide has the following formula (B):

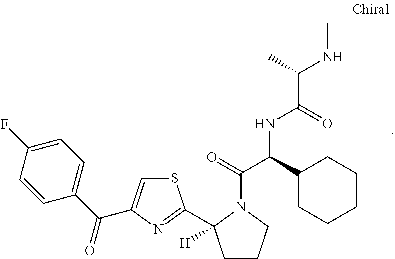

The term “delay of progression”, as used herein, means administration of the combination to patients being in an early phase of the proliferative disease to be treated.

Combination refers to administration of an amount of VDA in combination with administration of an amount of IAP antagonists such that there is a synergistic effect which would not be obtained if a VDA is administered without separate, simultaneous or sequential administration of IAP antagonists or an synergist effect which would not be obtained if there is administered IAP antagonists without the separate, simultaneous or sequential administration of a VDA, wherein administration can be continuous, sequential or sporadic.

Preferably, combination refers to administration of an amount of a VDA in combination with administration of an amount of IAP antagonists such that there is a synergistic antiproliferative effect and/or a clonogenic cell killing effect that would not be obtained if:

a) The VDA is administered without prior, simultaneous or subsequent administration of IAP antagonists. Wherein administration can be continuous, sequential or sporadic;
b) There is administration of IAP antagonists without the prior, simultaneous or subsequent administration of a VDA. Where in administration can be continuous, sequential or sporadic.
A combination which comprises:
(a) a VDA, which may be present in free form or in the form of a pharmaceutically acceptable salt and optionally at least one pharmaceutically acceptable carrier; and
(b) IAP antagonists, will be referred to hereinafter as a COMBINATION OF THE INVENTION.

In the combination of the invention, a VDA and pharmaceutically acceptable salts and prodrug derivatives are preferably used in the form of pharmaceutical preparations that contain the relevant therapeutically effective amount of active ingredient optionally together with or in admixture with inorganic or organic, solid or liquid, pharmaceutically acceptable carriers which are suitable for administration.

In an alternative embodiment, an IAP antagonist is given as a pre-treatment, i.e. before the treatment with the COMBINATION OF THE INVENTION is started; an IAP antagonist alone is administered to the patient for a defined period of time.

The VDA pharmaceutical compositions may be, e.g., compositions for enteral, such as oral, rectal, aerosol inhalation or nasal administration, compositions for parenteral, such as intravenous or subcutaneous administration, or compositions for transdermal administration (e.g., passive or iontophoretic), or compositions for topical administration.

Preferably, The VDA pharmaceutical compositions are adapted to oral administration.

The pharmaceutical compositions according to the invention can be prepared in a manner known per se and are those suitable for enteral, such as oral or rectal, and parenteral administration to mammals (warm-blooded animals), including man, comprising a therapeutically effective amount of at least one pharmacologically active combination partner alone or in combination with one or more pharmaceutically acceptable carries, especially suitable for enteral or parenteral application.

The novel pharmaceutical composition contain, e.g., from about 10% to about 100%, preferably from about 20% to about 60%, of the active ingredients. Pharmaceutical preparations for the combination therapy for enteral or parenteral administration are, e.g., those in unit dosage forms, such as sugar-coated tablets, tablets, capsules or suppositories, and furthermore ampoules. If not indicated otherwise, these are prepared in a manner known per se, e.g., by means of conventional mixing, granulating, sugar-coating, dissolving or lyophilizing processes. It will be appreciated that the unit content of a combination partner contained in an individual dose of each dosage form need not in itself constitute an effective amount since the necessary effective amount can be reached by administration of a plurality of dosage units.

In preparing the compositions for oral dosage form, any of the usual pharmaceutical media may be employed, such as, e.g., water, glycols, oils, alcohols, flavouring agents, preservatives, colouring agents; or carriers such as starches, sugars, microcrystalline cellulose, diluents, granulating agents, lubricants, binders, disintegrating agents and the like in the case of oral solid preparations, such as, e.g., powders, capsules and tablets, with the solid oral preparations being preferred over the liquid preparations. Because of their ease of administration, tablets and capsules represent the most advantageous oral dosage unit form in which case solid pharmaceutical carriers are obviously employed.

In particular, a therapeutically effective amount of each combination partner of the COMBINATION OF THE INVENTION may be administered simultaneously or sequentially and in any order, and the components may be administered separately or as a fixed combination. For example, the method of delay of progression or treatment of a proliferative disease according to the invention may comprise:

(i) administration of the first combination partner; and
(ii) administration of the second combination partner,
wherein administration of a combination partner may be simultaneous or sequential in any order, in jointly therapeutically effective amounts, preferably in synergistically effective amounts, e.g., in daily or weekly dosages corresponding to the amounts described herein. The individual combination partners of the COMBINATION OF THE INVENTION can be administered separately at different times during the course of therapy or concurrently. Furthermore, the term administering also encompasses the use of a pro-drug of a VDA that converts in vivo to the combination partner as such. The instant invention is therefore to be understood as embracing all such regimes of simultaneous or alternating treatment and the term “administering” is to be interpreted accordingly.

The dosage of an IAP antagonist and a VDA in relation to each other is preferably in a ratio that is synergistic.

If the warm-blooded animal is a human, the dosage of a compound of formula (I) is preferably an appropriate dose in the range from 100-1,500 mg daily, e.g., 200-1,000 mg/day, such as 200, 400, 500, 600, 800, 900 or 1,000 mg/day, administered in one or two doses daily. Appropriate dosages and the frequency of administration of the death receptor ligand will depend on such factors, as the nature and severity of the indication being treated, the desired response, the condition of the patient and so forth.

The particular mode of administration and the dosage of a VDA may be selected by the attending physician taking into account the particulars of the patient, especially age, weight, life style, activity level, etc.

The dosage of a VDA may depend on various factors, such as effectiveness and duration of action of the active ingredient, mode of administration, effectiveness and duration of action of the ionizing radiation and/or sex, age, weight and individual condition of the subject to be treated.

The dosage of ionizing radiation may depend on various factors, such as effectiveness and duration of action of the ionizing radiation, mode of administration, location of administration, effectiveness and duration of action of The VDA inhibitor and/or sex, age, weight and individual condition of the subject to be treated. The dosage of ionizing radiation is generally defined in terms of radiation absorbed dose, time and fraction, and must be carefully defined by the attending physician.

In one preferred embodiment of the invention the combination comprises an IAP antagonist and 5,6-dimethylxanthenone-4-acetic acid or a pharmaceutically acceptable salt thereof.

In another preferred embodiment of the invention, the combination comprises (S)—N—((S)-1-cyclohexyl-2-{(S)-2-[4-(4-fluoro-benzoyl)-thiazol-2-yl]-pyrrolidin-1-yl}-2-oxo-ethyl)-2-methylamino-propionamide and 5,6-dimethylxanthenone-4-acetic acid or a pharmaceutically acceptable salt thereof.

Moreover, the present invention relates to a method of treating a warm-blooded animal having a proliferative disease comprising administering to the animal a COMBINATION OF THE INVENTION in a way that is jointly therapeutically effective against a proliferative disease and in which the combination partners can also be present in the form of their pharmaceutically acceptable salts.

Furthermore, the present invention pertains to the use of a COMBINATION OF THE INVENTION for the delay of progression or treatment of a proliferative disease and for the preparation of a medicament for the delay of progression or treatment of a proliferative disease.

The following examples demonstrate that treatment with either 5,6-dimethylxanthenone-4-acetic acid or S)—N—((S)-1-cyclohexyl-2-{(S)-2-[4-(4-fluoro-benzoyl)-thiazol-2-yl]-pyrrolidin-1-yl}-2-oxo-ethyl)-2-methylamino-propionamide results in significant anti-tumor activity in the MDA-MB-231 breast tumor xenograft model. Treatment with S)—N—((S)-1-cyclohexyl-2-{(S)-2-[4-(4-fluoro-benzoyl)-thiazol-2-yl]-pyrrolidin-1-yl}-2-oxo-ethyl)-2-methylamino-propionamide results in increased levels of human TNF from the xenograft tissue while treatment with 5,6-dimethylxanthenone-4-acetic acid increased host (murine) TNF. The combination of S)—N—((S)-1-cyclohexyl-2-{(S)-2-[4-(4-fluoro-benzoyl)-thiazol-2-yl]-pyrrolidin-1-yl}-2-oxo-ethyl)-2-methylamino-propionamide with 5,6-dimethylxanthenone-4-acetic acid demonstrated improved anti-tumor activity compared to either single agent therapy alone. Similar combination benefit of S)—N—((S)-1-cyclohexyl-2-{(S)-2-[4-(4-fluoro-benzoyl)-thiazol-2-yl]-pyrrolidin-1-yl}-2-oxo-ethyl)-2-methylamino-propionamide and 5,6-dimethylxanthenone-4-acetic acid was also observed in a melanoma xenograft model. The following examples are merely illustrative and not meant to limit the scope of the present invention in any manner:

### Example 1

### Single Agent Anti-tumor Activity of (S)—N—((S)-1-cyclohexyl-2-{(S)-2-[4-(4-fluoro-benzoyl)-thiazol-2-yl]-pyrrolidin-1-yl}-2-oxo-ethyl)-2-methylamino-propionamide (Compound B) or 5,6-dimethylxanthenone-4-acetic acid (Compound A)

Treatment started on day 50 following orthotopic implantation of 7.5×10⁶ MDA-MB-231 human breast cancer cells into the mammary fat pad. Starting tumor volume averaged 160 mm³ (range=76 to 286); n=8/group. (S)—N—((S)-1-cyclohexyl-2-{(S)-2-[4-(4-fluoro-benzoyl)-thiazol-2-yl]-pyrrolidin-1-yl}-2-oxo-ethyl)-2-methylamino-propionamide was administered orally (po) at 150 mg/kg, qw on days 50, 57, and 64 post implantation. 5,6-dimethylxanthenone-4-acetic acid was administered intravenously (iv) at 20 mg/kg on days 50, 54, and 58 post implantation. Vehicle control for (S)—N—((S)-1-cyclohexyl-2-{(S)-2-[4-(4-fluoro-benzoyl)-thiazol-2-yl]-pyrrolidin-1-yl}-2-oxo-ethyl)-2-methylamino-propionamide is 30% 0.1N HCl and 70% 100 mM sodium acetate buffer, pH 4.63 (used in study). The vehicle for 5,6-dimethylxanthenone-4-acetic acid is 10% Tris-HCl buffer and 90% 1×PBS. Statistical analysis performed on delta tumor volumes on day 71 post implantation using a One-way ANOVA followed by a post hoc Tukey test. Both (S)—N—((S)-1-cyclohexyl-2-{(S)-2-[4-(4-fluoro-benzoyl)-thiazol-2-yl]-pyrrolidin-1-yl}-2-oxo-ethyl)-2-methylamino-propionamide and 5,6-dimethylxanthenone-4-acetic acid demonstrated single agent anti-tumor activity in the MDA-MB-231 human breast cancer orthotopic xenograft model in athymic nude mice as described in FIGS. 1A and 1B (p<0.05 compared to vehicle controls).

At the end of the study, tumors were collected 6 hrs after a final dose of (S)—N—((S)-1-cyclohexyl-2-{(S)-2-[4-(4-fluoro-benzoyl)-thiazol-2-yl]-pyrrolidin-1-yl}-2-oxo-ethyl)-2-methylamino-propionamide or 5,6-dimethylxanthenone-4-acetic acid. Treatment with (S)—N—((S)-1-cyclohexyl-2-{(S)-2-[4-(4-fluoro-benzoyl)-thiazol-2-yl]-pyrrolidin-1-yl}-2-oxo-ethyl)-2-methylamino-propionamide shows an increase in human TNF in MDA-MB-231 tumor lysates as described in FIG. 2. Treatment with 5,6-dimethylxanthenone-4-acetic acid shows an increase in mouse TNF as described in FIG. 3. Due to the small sample size (n=4), statistical analysis was not performed.

### Example 2

### Combination Activity of (S)—N—((S)-1-cyclohexyl-2-{(S)-2-[4-(4-fluoro-benzoyl)-thiazol-2-yl]-pyrrolidin-1-yl}-2-oxo-ethyl)-2-methylamino-propionamide (Compound B) with 5,6-dimethylxanthenone-4-acetic acid (Compound A) in MDA-MB-231 Human Breast Xenograft

Treatment started on day 38 following orthotopic implantation of 7.5×10⁶ MDA-MB-231 human breast cancer cells into the mammary fat pad. Starting tumor volume averaged 147 mm³ (range=90 to 288); n=8/group. (S)—N—((S)-1-cyclohexyl-2-{(S)-2-[4-(4-fluoro-benzoyl)-thiazol-2-yl]-pyrrolidin-1-yl}-2-oxo-ethyl)-2-methylamino-propionamide was administered orally (po) at 100 mg/kg, qw on days 38, 45, and 55 post implantation. 5,6-dimethylxanthenone-4-acetic acid was administered intravenously (iv) at 15 mg/kg on days 38, 42, and 46 post implantation. The combination of (S)—N—((S)-1-cyclohexyl-2-{(S)-2-[4-(4-fluoro-benzoyl)-thiazol-2-yl]-pyrrolidin-1-yl}-2-oxo-ethyl)-2-methylamino-propionamide and 5,6-dimethylxanthenone-4-acetic acid are evaluated in this study. Vehicle control for (S)—N—((S)-1-cyclohexyl-2-{(S)-2-[4-(4-fluoro-benzoyl)-thiazol-2-yl]-pyrrolidin-1-yl}-2-oxo-ethyl)-2-methylamino-propionamide is 30% 0.1N HCl and 70% 100 mM sodium acetate buffer, pH 4.63. Vehicle control for 5,6-dimethylxanthenone-4-acetic acid is 10% 1×Tris-HCl and 90% 1×PBS. Statistical analysis performed on tumor volumes from day 42-62 post implantation using a repeated measure MANOVA test. The combination of (S)—N—((S)-1-cyclohexyl-2-{(S)-2-[4-(4-fluoro-benzoyl)-thiazol-2-yl]-pyrrolidin-1-yl}-2-oxo-ethyl)-2-methylamino-propionamide and 5,6-dimethylxanthenone-4-acetic acid resulted in a 7% regression as described in FIG. 4A (p<0.05 compared to all groups). All treatments were well tolerated as monitored body weight change as described in FIG. 4B.

### Example 3

### Efficacy of (S)—N—((S)-1-cyclohexyl-2-{(S)-2-[4-(4-fluoro-benzoyl)-thiazol-2-yl]-pyrrolidin-1-yl}-2-oxo-ethyl)-2-methylamino-propionamide (Compound B) and 5,6-dimethylxanthenone-4-acetic acid (Compound A) following Concurrent Dosing in MDA-MB-231 Human Breast xenograft

To leverage the host TNF induction observed following treatment with 5,6-dimethylxanthenone-4-acetic acid, a concurrent dosing regimen was investigated in a repeat study in the MDA-MB-231 human breast xenograft model. Treatment started on day 26 following subcutaneous implantation of 5.0×10⁶ MDA-MB-231 human breast cancer cells in 50% basement membrane matrix. Starting tumor volume averaged 180 mm³ (range=120 to 231); n=12/group. In this study, (S)—N—((S)-1-cyclohexyl-2-{(S)-2-[4-(4-fluoro-benzoyl)-thiazol-2-yl]-pyrrolidin-1-yl}-2-oxo-ethyl)-2-methylamino-propionamide was administered orally (po) at 100 mg/kg, qw, 5,6-dimethylxanthenone-4-acetic acid was administered intravenously (iv) at 15 mg/kg, or (S)—N—((S)-1-cyclohexyl-2-{(S)-2-[4-(4-fluoro-benzoyl)-thiazol-2-yl]-pyrrolidin-1-yl}-2-oxo-ethyl)-2-methylamino-propionamide and 5,6-dimethylxanthenone-4-acetic acid were administered in combination on days 26, 33, and 40 post implantation. Vehicle control for (S)—N—((S)-1-cyclohexyl-2-{(S)-2-[4-(4-fluoro-benzoyl)-thiazol-2-yl]-pyrrolidin-1-yl}-2-oxo-ethyl)-2-methylamino-propionamide is 30% 0.1N HCl and 70% 100 mM sodium acetate buffer, pH 4.63. Vehicle control for 5,6-dimethylxanthenone-4-acetic acid is 10% 1×Tris-HCl and 90% 1×PBS. At the end of study, there were 11/12 surviving animals in the (S)—N—((S)-1-cyclohexyl-2-{(S)-2-[4-(4-fluoro-benzoyl)-thiazol-2-yl]-pyrrolidin-1-yl}-2-oxo-ethyl)-2-methylamino-propionamide treated group and 10/12 surviving animals in the combination group. Statistical analysis performed on tumor volumes from day 25-48 post implantation using a repeated measures MANOVA test. The combination of (S)—N—((S)-1-cyclohexyl-2-{(S)-2-[4-(4-fluoro-benzoyl)-thiazol-2-yl]-pyrrolidin-1-yl}-2-oxo-ethyl)-2-methylamino-propionamide and 5,6-dimethylxanthenone-4-acetic acid resulted in a 30% regression (p<0.05 compared to all groups) as described in FIG. 5A. As shown in FIG. 5B, there was body weight loss following treatment that recovered during the dosing interval

### Example 4

### Combination Activity of (S)—N—((S)-1-cyclohexyl-2-{(S)-2-[4-(4-fluoro-benzoyl)-thiazol-2-yl]-pyrrolidin-1-yl}-2-oxo-ethyl)-2-methylamino-propionamide (Compound B) with 5,6-dimethylxanthenone-4-acetic acid (Compound A) in HS944.t Human Melanoma Xenograft

Treatment started on day 14 following subcutaneous implantation of 5.0×10⁶ Hs944.t human melanoma cancer cells in 50% basement membrane matrix. Starting tumor volume averaged 245 mm³ (range=146 to 321); n=8/group. In this study, (S)—N—((S)-1-cyclohexyl-2-{(S)-2-[4-(4-fluoro-benzoyl)-thiazol-2-yl]-pyrrolidin-1-yl}-2-oxo-ethyl)-2-methylamino-propionamide was administered orally (po) at 100 mg/kg, qw, 5,6-dimethylxanthenone-4-acetic acid was administered intravenously (iv) at 15 mg/kg, or (S)—N—((S)-1-cyclohexyl-2-{(S)-2-[4-(4-fluoro-benzoyl)-thiazol-2-yl]-pyrrolidin-1-yl}-2-oxo-ethyl)-2-methylamino-propionamide and 5,6-dimethylxanthenone-4-acetic acid were administered in combination on days 14 and 21 post implantation. Vehicle control for (S)—N—((S)-1-cyclohexyl-2-{(S)-2-[4-(4-fluoro-benzoyl)-thiazol-2-yl]-pyrrolidin-1-yl}-2-oxo-ethyl)-2-methylamino-propionamide is 30% 0.1N HCl and 70% 100 mM sodium acetate buffer, pH 4.63. Vehicle control for 5,6-dimethylxanthenone-4-acetic acid is 10% 1×Tris-HCl and 90% 1×PBS. Statistical analysis performed on tumor volumes from day 14-27 post implantation using a repeated measures MANOVA test. At study termination, there were 7/8 surviving animals in the (S)—N—((S)-1-cyclohexyl-2-{(S)-2-[4-(4-fluoro-benzoyl)-thiazol-2-yl]-pyrrolidin-1-yl}-2-oxo-ethyl)-2-methylamino-propionamide and 5,6-dimethylxanthenone-4-acetic acid single agent and combination groups. The combination of (S)—N—((S)-1-cyclohexyl-2-{(S)-2-[4-(4-fluoro-benzoyl)-thiazol-2-yl]-pyrrolidin-1-yl}-2-oxo-ethyl)-2-methylamino-propionamide and 5,6-dimethylxanthenone-4-acetic acid resulted in a T/C=16% as shown in FIG. 6A (p

<0.05 compared to all groups). The body weight change is illustrated in FIG. 6B.

1. A method for treating a proliferative disease in a subject in need of such treatment, wherein the method comprises administering:
(a) a vascular disrupting agent, in combination with (b) an IAP antagonist, or a pharmaceutically acceptable salt thereof.

2. A method for treating a proliferative disease in a subject in need of such treatment, wherein the method comprises administering:
(a) a vascular disrupting agent having the following formula (A): 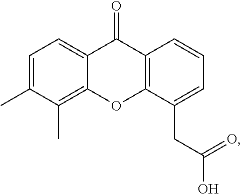
or a pharmaceutically acceptable salt thereof, in combination with
(b) an IAP antagonist of the following formula I: 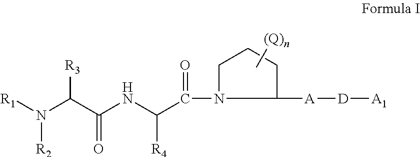
or pharmaceutically acceptable salts thereof, wherein
R₁ is H, C₁-C₄ alkyl, C₂-C₄ alkenyl, C₂-C₄ alkynyl or C₃-C₁₀ cycloalkyl, which R₁ may be unsubstituted or substituted;
R₂ is H, C₁-C₄ alkyl, C₂-C₄ alkenyl, C₂-C₄ alkynyl, C₃-C₁₀ cycloalkyl which R₂ may be unsubstituted or substituted;
R₃ is H, CF₃, C₂F₅, C₁-C₄ alkyl, C₂-C₄ alkenyl, C₂-C₄ alkynyl, CH₂—Z or R₂ and R₃ taken together with the nitrogen atom to which they are attached form a heterocyclic ring, which alkyl, alkenyl, alkynyl or het ring may be unsubstituted or substituted;
Z is H, OH, F, Cl, CH₃, CH₂Cl, CH₂F or CH₂OH;
R₄ is C₀₋₁₀ alkyl, C₃-C₁₀ cycloalkyl, wherein the C₀₋₁₀ alkyl, or cycloalkyl group is unsubstituted or substituted;
A is het, which may be substituted or unsubstituted;
D is C₁-C₇ alkylene or C₂-C₉ alkenylene, C(O), O, NR₇, S(O)r, C(O)—C₁-C₁₀ alkyl, O—C₁-C₁₀ alkyl, S(O)r-C₁-C₁₀ alkyl, C(O)C₀₋₁₀ arylalkyl OC₀₋₁₀ arylalkyl, or S(O)r C₀₋₁₀ arylalkyl, which alkyl and aryl groups may be unsubstituted or substituted;
r is 0, 1, or 2;
A₁ is a substituted aryl or unsubstituted or substituted het which substituents on aryl and het are halo, lower alkoxy, NR₅R₆, CN, NO₂ or SR₅;
Each Q is independently H, C₁-C₁₀ alkyl, C₁-C₁₀ alkoxy, aryl C₁-C₁₀ alkoxy, OH, O—C₁-C₁₀-alkyl, (CH₂)₀₋₆—C₃-C₇ cycloalkyl, aryl, aryl C₁-C₁₀ alkyl, O—(CH₂)₀₋₆aryl, (CH₂)₁₋₆het, het, O—(CH₂)₁₋₆het, —OR₁₁, C(O)R₁₁, —C(O)N(R₁₁)(R₁₂), N(R₁₁)(R₁₂), SR₁₁, S(O)R₁₁,S(O)₂R₁₁, S(O)₂—N(R₁₁)(R₁₂), or NR₁₁—S(O)₂—(R₁₂), wherein alkyl, cycloalkyl and aryl are unsubstituted or substituted;
n is 0, 1, 2 or 3, 4, 5, 6 or 7;
“Het” is a 5-7 membered monocyclic heterocyclic ring containing 1-4 heteroring atoms selected from N,O and S or an 8-12 membered fused ring system that includes one 5-7 membered monocyclic heterocyclic ring containing 1, 2, or 3 heteroring atoms selected from N, O and S, which het is unsubstituted or substituted;
R₁₁ and R₁₂ are independently H, C₁-C₁₀ alkyl, (CH₂)₀₋₆—C₃-C₇cycloalkyl, (CH₂)₀₋₆—(CH)₀₋₁(aryl)₁₋₂,C(O)—C₁-C₁₀alkyl, —C(O)—(CH₂)₁₋₆—C₃-C₇cycloalkyl, —C(O)—O—(CH₂)₀₋₆-aryl, —C(O)—(CH₂)₀₋₆—O-fluorenyl, C(O)—NH—(CH₂)₀₋₆-aryl, C(O)—(CH₂)₀₋₆-aryl, C(O)—(CH₂)₁₋₆-het, —C(S)—C₁-C₁₀alkyl, —C(S)—(CH₂)₁₋₆—C₃-C₇cycloalkyl, —C(S)—O—(CH₂)₀₋₆-aryl, —C(S)—(CH₂)₀₋₆—O-fluorenyl, C(S)—NH—(CH₂)₀₋₆-aryl, —C(S)—(CH₂)₀₋₆-aryl or C(S)—(CH₂)₁₋₆-het, C(O)R₁₁, C(O)NR₁₁R₁₂, C(O)OR₁₁, S(O)nR₁₁, S(O)ₘNR₁₁R₁₂, m=1 or 2, C(S)R₁₁, C(S)NR₁₁R₁₂, C(S)OR₁₁, wherein alkyl, cycloalkyl and aryl are unsubstituted or substituted; or R₁₁ and R₁₂ are a substituent that facilitates transport of the molecule across a cell membrane; or R₁₁ and R₁₂ together with the nitrogen atom form het;
wherein the alkyl substituents of R₁₁ and R₁₂ may be unsubstituted or substituted by one or more substituents selected from C₁-C₁₀alkyl, halogen, OH, O—C₁-C₆alkyl, —S—C₁-C₆alkyl, CF₃ or NR₁₁R₁₂;
Substituted cycloalkyl substituents of R₁₁ and R₁₂ are substituted by one or more substituents selected from a C₂-C₁₀ alkene; C₁-C₆alkyl; halogen; OH; O—C₁-C₆alkyl; S—C₁-C₆alkyl, CF₃; or NR₁₁R₁₂ and
Substituted het or substituted aryl of R₁₁ and R₁₂ are substituted by one or more substituents selected from halogen, hydroxy, C₁-C₄ alkyl, C₁-C₄ alkoxy, nitro, CN O—C(O)—C₁-C₄alkyl and C(O)—O—C₁-C₄-alkyl;
R₅, R₆ and R₇ are independently hydrogen, lower alkyl, aryl, aryl lower alkyl, cycloalkyl, or cycloalkyl lower alkyl, and
wherein the substituents on R₁, R₂, R₃, R₄, Q, and A and A₁ groups are independently halo, hydroxy, lower alkyl, lower alkenyl, lower alkynyl, lower alkanoyl, lower alkoxy, aryl, aryl lower alkyl, amino, amino lower alkyl, diloweralkylamino, lower alkanoyl, amino lower alkoxy, nitro, cyano, cyano lower alkyl, carboxy, lower carbalkoxy, lower alkanoyl, aryloyl, lower arylalkanoyl, carbamoyl, N-mono- or N,N-dilower alkyl carbamoyl, lower alkyl carbamic acid ester, amidino, guanidine, ureido, mercapto, sulfo, lower alkylthio, sulfoamino, sulfonamide, benzosulfonamide, sulfonate, sulfanyl lower alkyl, aryl sulfonamide, halogen substituted aryl sulfonate, lower alkylsulfinyl, arylsulfinyl; aryl-lower alkylsulfinyl, lower alkylarylsulfinyl, lower alkylsulfonyl, arylsulfonyl, aryl-lower alkylsulfonyl, lower aryl alkyl lower alkylarylsulfonyl, halogen-lower alkylmercapto, halogen-lower alkylsulfonyl, phosphono (—P(═O)(OH)₂), hydroxy-lower alkoxy phosphoryl or di-lower alkoxyphosphoryl, (R₉)NC(O)—NR₁₀R₁₃, lower alkyl carbamic acid ester or carbamates or —NR₈R₁₄, wherein R₈ and R₁₄ can be the same or different and are independently H or lower alkyl, or R₈ and R₁₄ together with the N atom form a 3- to 8-membered heterocyclic ring containing a nitrogen heteroring atoms and may optionally contain one or two additional heteroring atoms selected from nitrogen, oxygen and sulfur, which heterocyclic ring may be unsubstituted or substituted with lower alkyl, halo, lower alkenyl, lower alkynyl, hydroxy, lower alkoxy, nitro, amino, lower alkyl, amino, diloweralkyl amino, cyano, carboxy, lower carbalkoxy, formyl, lower alkanoyl, oxo, carbarmoyl, N-lower or N,N-dilower alkyl carbamoyl, mercapto, or lower alkylthio, and
R₉, R₁₀, and R₁₃ are independently hydrogen, lower alkyl, halogen substituted lower alkyl, aryl, aryl lower alkyl, halogen substituted aryl, halogen substituted aryl lower alkyl.

3. The method of claim 1 wherein the IAP antagonist is described by formula (B): 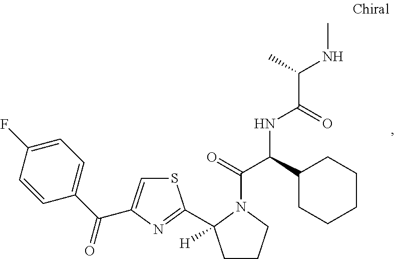
or a pharmaceutically acceptable salt thereof.

4. The method according to claim 1, wherein the proliferative disease is breast cancer or melanoma.

5. (canceled)

6. (canceled)

7. A package comprising a compound of formula (A), 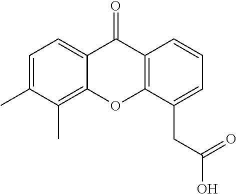
or a pharmaceutically acceptable salt thereof,
in pharmaceutically acceptable form, together with instructions for the use in combination with a or a pharmaceutically acceptable salt thereof compound of formula (B) 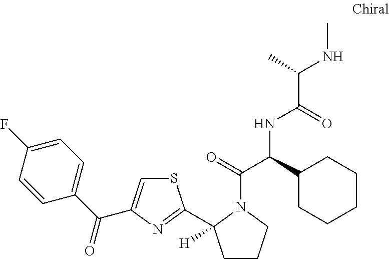
for the treatment of a proliferative disease.

8. The package according to claim 7 wherein the proliferative disease is breast cancer or melanoma.

9. A method of treating a patient suffering from breast cancer comprising administering an effective amount of (S)—N—((S)-1-cyclohexyl-2-{(S)-2-[4-(4-fluoro-benzoyl)-thiazol-2-yl]-pyrrolidin-1-yl}-2-oxo-ethyl)-2-methylamino-propionamide.

10. A method of treating a patient suffering from breast cancer comprising administering an effective amount of 5,6-dimethylxanthenone-4-acetic acid.
